(19) United States
(12) Patent Application Publication
(10) Pub. No.: US 2003/0129740 A1
(43) Pub. Date: Jul. 10, 2003

(54) Method of preparing substrate having functional group pattern for immobilizing physiological material

(76) Inventors: Kang-Il Seo, Suwon-city (KR); Hun-Soo Kim, Seoul (KR); Ji-Na Namgoong, Yongin-city (KR); Eun-Keu Oh, Suwon-city (KR); In-Ho Lee, Incheon-city (KR); Young-Do Choi, Boocheon-city (KR); Tai-Jun Park, Seoul (KR)

Correspondence Address: CHRISTIE, PARKER & HALE, LLP, P.O. BOX 7068, PASADENA, CA, 91109-7068, US

(21) Appl. No.: 10/337,927

(22) Filed: Jan. 7, 2003

(30) Foreign Application Priority Data
Jan. 7, 2002 (KR) 2002-789

(51) Int. Cl. B05D 3/00; C12M 1/34

(52) U.S. Cl. 435/287.2; 427/2.11

(57) ABSTRACT

A method for preparing a patterned substrate for immobilizing a physiological material is provided. The patterned substrate comprises a primer layer formed on a substrate for controlling surface tension of the upper layer of the immobilization layer, wherein the primer layer has reactive groups to bind an immobilization functional group and hydrophobic functional groups and thus is capable of providing a functional group pattern. The substrate for immobilizing a physiological material can provide the immobilization layer with a stable, uniform, and high-density functional group pattern through a simple process.

### CROSS-REFERENCE TO RELATED APPLICATION

This application claims priority of Korean Patent Application No. 2002-789, filed Jan. 7, 2002, the entire disclosure of which is incorporated herein by reference.

### FIELD OF THE INVENTION

The present invention relates to a method of preparing a patterned substrate for immobilizing a physiological material, and more particularly, to a method of preparing a substrate having an immobilization functional group pattern with a uniform distribution and a high density for immobilizing a physiological material.

### BACKGROUND OF THE INVENTION

Recently, demands for the development of technology used for analyzing the activity of physiological materials, such as nucleic acids, proteins, enzymes, antibodies, and antigens, have rapidly increased in the world. For such demands, a biochip in which the required physiological material molecules are immobilized on certain tiny regions by adopting semiconductor processing techniques is suggested, so that physiologically useful information is easily obtained just by bio-chemically searching the biochip.

The biochip is in the form of a conventional semiconductor chip, but integrated thereon is a bio-organic material such as an enzyme, a protein, an antibody, DNA, a microorganism, animal and/or plant cells and/or organs, a neuron, or the like. The biochip can be classified as a “DNA chip” immobilizing a DNA probe, a “protein chip” immobilizing a protein such as an enzyme, an antibody, an antigen or the like, or a “lab-on-a-chip” which is integrated with pre-treating, biochemical reacting, detecting, and data-analyzing functions to impart an auto-analysis function.

The biochip is a device used for diagnosing infectious diseases and analyzing genes by using an intrinsic function of physiological material and a mimicking function of a living body. It has recently become noteworthy as an essential device of a bio-computer which recognizes and responds to foreign stimulation like a living body and has a superior capacity to currently commercialized semiconductors.

To achieve the successful development of such a biochip, it is important to find a method for immobilizing a physiological material in which an interface between the physiological material and a substrate is efficiently formed, and wherein the inherent functions of the physiological material can be utilized at a maximum level. Generally, the physiological material is immobilized on the surface of a glass plate, a silicon wafer, a microwell plate, a tube, a spherical bead, a surface with a porous layer, etc. by various techniques, for example, by reacting DNA with carbodiimide to activate a 5′-phosphate group of DNA, and by reacting the activated DNA with a functional group on the surface of the substrate so as to immobilize the DNA on the substrate.

U.S. Pat. No. 5,858,653 discloses a composition comprising an ion group, such as a quaternary ammonium group, a protonated tertiary amine, or phosphonium, capable of reacting with a target physiological material, and a polymer having a photo-reactive group or a thermochemically reactive group for use in attaching to the surface of a substrate. U.S. Pat. No. 5,981,734 teaches that when DNA is immobilized by a polyacrylamide gel having an amino group or an aldehyde group, the DNA can be bound with a substrate via a stable hybridization bond to easily facilitate carrying out of analysis. U.S. Pat. No. 5,869,272 discloses an attachment layer comprising a chemical selected from dendrimers, star polymers, molecular self-assembling polymers, polymeric siloxanes, and film-forming latexes formed by spin-coating a silicone wafer with aminosilane. U.S. Pat. No. 5,869,272 also discloses a method for the determination of a bacteria antigen by detecting a visual color change of an optically active surface. U.S. Pat. No. 5,919,523 discloses a method for preparing a support on which an amino silane-treated substrate is doped with glycine or serine or is coated with an amine, imine, or amide-based organic polymer.

In the above-mentioned patents, the immobilization layer is provided by preparing a self-assembly monolayer of silane molecules. Preferably, the silane is aminoalkoxy silane since it does not produce acidic by-products, and it can provide a molecular layer having a functional group with a relatively high density. Although much research has advanced the obtainment of a uniform monolayer having high-density functional groups using aminoalkoxy silanes, an aminosilane monolayer having a functional group with a uniform and high density and shorter manufacturing time has not been achieved.

U.S. Pat. No. 5,985,551 discloses a method for providing amino groups on a solid substrate by using a photolithography technique on the amino silane treated substrate, the method involving allotting hydrophilic functions on regions to immobilize DNA and fluorosiloxane hydrophobic functions on other regions so as to form a desirable patterned DNA spot on the substrate. This method is advantageous for controlling density of the functional groups by separating immobilizing regions from non-immobilizing regions. However, it has a problem in that the process is very complicated with its multiple steps, and it has a longer manufacturing time and thus is inadequate for large-scale production.

### SUMMARY OF THE INVENTION

The present invention provides a method of preparing a functional group patterned substrate for immobilizing a physiological material comprising: a) preparing a coating composition including an alkoxide compound and a hydrophobic functionalized silane compound; b) coating the composition on a substrate to form a primer layer for controlling the surface tension of an immobilization layer; c) forming an immobilization functional group pattern by coating a composition including a compound having a functional group capable of immobilizing the physiological material on the primer-layer-coated substrate to prepare a patterned substrate; and d) subjecting the patterned substrate to heat-treatment.

The present invention further provides a functional-group-patterned substrate comprising a) a substrate; b) a primer layer formed on the substrate for controlling the surface tension of the upper layer of the immobilization layer, wherein the primer layer has reactive groups to bind with immobilization functional groups and hydrophobic functional groups capable of controlling functional group patterning; and c) a patterned immobilization layer formed on the primer layer for immobilizing the physiological material.

The present invention also provides a biochip comprising a physiological material immobilized on the surface of the patterned substrate.

### BRIEF DESCRIPTION OF THE DRAWINGS

A more complete appreciation of the invention, and many of the attendant advantages thereof, will be readily apparent as the same becomes better understood by reference to the following detailed description when considered in conjunction with the accompanying drawings, wherein:

FIG. 1 is a schematic diagram illustrating a process of fabricating a substrate for immobilizing a physiological material according to the present invention;

FIG. 2 is a cross-sectional view showing a conventional self-assembly monolayer;

FIG. 3 is a cross-sectional view showing a substrate for immobilizing a physiological material having a three-dimensional cross-linking structure according to the present invention; and

FIGS. 4A and 4B are photographs showing the functional-group-patterned substrate for immobilizing a physiological material according to Examples 1 and 2, respectively, of the present invention.

### DETAILED DESCRIPTION OF THE PREFERRED EMBODIMENTS

Hereinafter, the present invention is described in further detail.

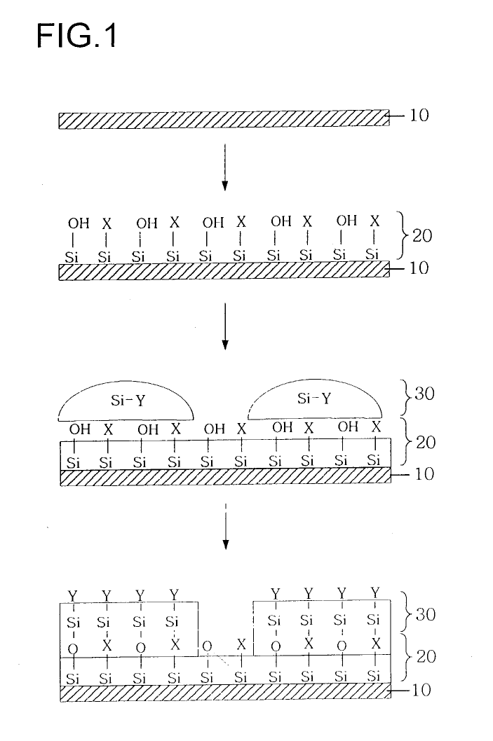

FIG. 1 is a schematic diagram illustrating a process of fabricating a substrate for immobilizing a physiological material. As shown in FIG. 1, the substrate for immobilizing a physiological material includes a substrate 10, and a primer layer 20 for controlling the surface tension of the upper layer of the immobilization layer, wherein the primer layer exists between the substrate 10 and an immobilization layer 30. The primer layer 20 is capable of controlling immobilization functional group patterning. It also includes a highly reactive group capable of binding with the immobilizing functionalized compound, so it imparts uniform arraying of the high-density functional group.

The substrate 10 of the present invention is exemplified by, but is not limited to, glass, a silicone wafer, polycarbonate, polystyrene, polyurethane, and the like. It may also be in a form of a microwell plate, a tube, a spherical bead, or a porous layer.

The primer layer capable of controlling the surface tension of the upper layer of the immobilization layer is formed from a coating composition comprising an alkoxide compound and a hydrophobic functionalized silane compound. The alkoxide compound is represented by the following formula (1):

M(OR¹)ₖ (1)

wherein

M is an element selected from the group consisting of 4B, 3A, 4A, and 5A group elements of the Periodic Table, and is preferably selected from the group consisting of Si, Zr, Ti, Al, Sn, In, and Sb;

R¹ is hydrogen or a C₁₋₂₀ alkyl or C₆₋₁₂ aromatic group, and is preferably selected from the group consisting of hydrogen, methyl, ethyl, propyl, butyl, and phenyl; and

k is a value ranging from 3 to 4, and is determined depending upon the valence of M.

The hydrophobic functionalized silane compound is represented by the following formula (2):

X—Si(R²)₃ (2)

wherein

X is a hydrophobic functional group, preferably a C₁₋₂₀ alkyl, a C₁₋₂₀ haloalkyl or C₆₋₁₂ aromatic group, and is more preferably methyl, octyl, heptadecafluoro-1,1,2,2-tetrahydrodecyl, (3-heptafluoroisopropoxy)propyl, or phenyl; and

R² is hydrogen, C₁₋₂₀ alkyl, or a halogen.

A preferred example of a compound represented by formula (1) is a silicon tetraalkoxide, such as tetraethyl orthosilicate, aluminum tributoxide, zirconium tetrabutoxide, and the like.

An example of the compound represented by formula (2) are (heptadecafluoro-1,1,2,2-tetrahydrodecyl)trialkoxysilanes such as (heptadecafluoro-1,1,2,2-tetra-hydrodecyl)triethoxysilane, (heptadecafluoro-1,1,2,2-tetrahydrodecyl)trichlorosilane, (3-heptafluoroisopropoxy)propyl trichlorosilane, and the like.

The compounds of formula (1) and formula (2) are preferably used in a mixed weight ratio of 99.999:0.001 to 50:50, more preferably from 99.9:0.1 to 90:10. When the amount of the alkoxide compound of formula (1) is more than 99.999 wt %, the controlling effect of the surface tension is insufficient. When the amount of the alkoxide compound of formula (1) is less than 50 wt %, the reactive groups to bind the immobilizing functional group are not sufficient and thus not preferable.

The coating composition further comprises the compound of the following formula (3) in addition to compounds of the formulas (1) and (2):

[M′(OR³)ₘ]ₚ(R⁴)_{q} (3)

wherein

M′ is an element selected from the group consisting of 4B, 3A, 4A, and 5A group elements of the Periodic Table, and is preferably selected from the group consisting of Si, Zr, Ti, Al, Sn, In, and Sb;

R³ is hydrogen, halogen, or C₁₋₂₀ alkyl or C₆₋₁₂ aromatic group, and is preferably hydrogen, chlorine, or methyl, ethyl, propyl, butyl, or phenyl;

R⁴ is methylene or phenyl, optionally substituted with a C₁₋₆ substituent;

m is a value ranging from 2 to 3 and is determined depending upon the valence of M′;

p is a numerical value ranging from 2 to 4; and

q is a numerical value ranging from 1 to 20.

The compound of formula (3) is contained in the primer layer, and thus blocks deposition of alkaline material from the substrate of a sodium lime glass and is capable of improving the attachment between the substrate 10 and the immobilizing functional group of the immobilization layer 30.

Examples of the compound represented by formula (3) are bis (triethoxysilyl)ethane, bis(trimethoxysilyl)hexane, bis(triethyoxysilyl)methane, 1,9-bis-(trichlorosilyl)nonane, bis(tri-n-butoxytin)methane, bis(triisopropoxytitanium)hexane, 1,4-bis(trimethoxysilylethyl)benzene, and the like.

The compound of the formula (3) is preferably used in an amount of 0.001 to 50 wt %, more preferably 0.01 to 10 wt %, based on the amount of the coating composition.

The primer layer is formed by coating a coating composition comprising compounds of the formulas (1) and (2), and optionally the compound of the formula (3), on the substrate. The coating composition is prepared by dissolving the compounds of the formulas (1) and (2), and optionally the compound of the formula (3), in a dilution solvent.

The dilution solvent is a mixture of water and an organic solvent, and the organic solvent is preferably an alcohol solvent such as methanol, ethanol, propanol, or butanol; a cellosolve solvent such as methyl cellosolve; any organic solvent compatible with water such as acetones; or any mixture thereof.

The compounds of formulas (1) and (2) and optionally the compound of formula (3) for forming the primer layer are dissolved in the solvent and form an oligomer via a hydrolysis reaction and a condensation reaction. In order to increase the reaction rate, any organic or inorganic acid, such as acetic acid, nitric acid, hydrochloric acid, or the like, is added so that the pH of the coating composition is adjusted to from 2 to 10.

The coating composition comprises the compounds of formulas (1) and (2) for forming the primer layer in an amount of from 0.1 to 90 wt %, preferably from 1 to 50 wt %. When the amount of the compounds is less than 0.1 wt %, the controlling capability of surface tension of the upper layer of the immobilization layer is not sufficient, whereas when it is more than 90 wt %, the coating composition cannot be applied to the substrate.

The primer layer is simply prepared by coating the coating composition on the substrate using a coating method. An example of the coating method includes, but is not limited to, a wet coating method such as dipping, spraying, spin-coating, or printing. In the present invention, the functional group pattern is formed by a relatively simpler process than in the U.S. Pat. No. 5,985,551 that uses photolithography.

As shown in FIG. 1, the primer layer 20 provides silanol groups (SiOH) that are capable of binding with an immobilization functional group, and hydrophobic functional groups (Si—X) that are present among the silanol groups and are capable of controlling the surface tension of the upper layer of the immobilization layer.

The silanol groups of the primer layer 20 provide regions for binding with an immobilization functional group to form a desirable immobilization functional group pattern, and the hydrophobic functional groups (Si—X) stably maintain the immobilization functional group pattern. The immobilization functional group pattern has a contact angle of 60 degrees or above, preferably 90 degrees or above.

The silanol groups bind with the silanol group of the immobilization functional compound through subsequent heat-treatment to form siloxane bonds (Si—O—Si). The siloxane bond between the primer layer and immobilization layer is stronger than the bond formed between the substrate and the immobilization functional compound. Therefore, the primer layer improves the attachment of the physiological materials. The siloxane group formed between silanol groups of the primer layer does not further react with physiological materials to be immobilized and thus improves the detecting performance of the bio-chip.

The immobilization layer is obtained by applying the compound comprising an immobilization functional group on the surface of the primer layer so that the substrate for immobilizing a physiological material is provided. Herein, the term “immobilization layer” means the coating layer of any compound having immobilization functional groups used in immobilizing the physiological material.

The immobilization functional group is exemplified by, but is not limited to, an amino, an aldehyde, a mercapto, or a carboxyl group. The compound having the immobilization group may be represented by the following formula (4).

Y-R⁵—Si(R⁶)₃ (4)

wherein

Y varies depending upon the terminal group of the physiological material and is at least one functional group selected from the group consisting of amino, aldehyde, mercapto, and carboxyl groups;

R⁵ is selected from the group consisting of C₁₋₂₀ alkyl groups, C₆₋₂₀ aromatic groups, ester groups, and imine groups, and is preferably a methyl group, an ethyl group, a propyl group, or a butyl group; and

R⁶ is selected from the group consisting of hydroxyl groups, C₁₋₂₀ alkoxy groups, acetoxy groups, halogen groups, and combinations thereof, and is preferably a hydroxy, methoxy, ethoxy, or acethoxy group.

Preferred examples of the compound of formula (4) having an amino group as the immobilization functional group include 3-aminopropyltrimethoxysilane, 3-aminopropyltriethoxysilane, 2-aminoundecyltrimethoxysilane, aminophenyltrimethoxy-silane, and N-(2-aminoethylaminopropyl)trimethoxysilane. The compound having the mercapto group is preferably exemplified by 3-mercaptopropyltrimethoxysilane, 3-mercaptopropyltriethoxysilane, etc. The compound having the aldehyde group is preferably exemplified by 4-trimethoxysilylbutanal, 4-trimethoxysilylbutanal, etc. The compound having the carboxyl group is preferably exemplified by carboxymethyltrimethoxysilane, carboxymethyltriethoxysilane, etc.

In order to reduce the hydrophilicity of the immobilization group and to improve the thermal stability of the three-dimensional cross-linking structure, the compound of formula (4) may be mixed with a hydrophobic silane compound represented by the following formula (5):

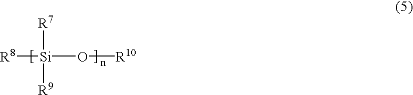

wherein

R⁷ is selected from the group consisting of C₁₋₁₄ alkyl groups, C₆₋₁₂ aromatic groups optionally substituted with preferably methyl, ethyl, or propyl, and CX₃, wherein X is a halogen, and preferably is methyl, ethyl, or propyl;

R⁸ and R⁹ are each independently selected from the group consisting of C₁₋₁₄ alkoxy groups, acetoxy groups, hydroxyl groups, and halogen groups, and preferably is methoxy, ethoxy, acetoxy or chlorine;

R₁₀ is selected from the group consisting of hydrogen, C₁₋₁₄ alkyl groups, and C₆₋₁₂ aromatic groups, and preferably is methyl or ethyl; and

n is an integer ranging from 1 to 15.

The hydrophilicity, the efficiency, the amount, and the shape of the immobilization layer can be controlled by adding the hydrophobic silane compound of the formula (5) to the compound having the immobilization functional group. The compound of the hydrophobic functional group having formula (2) described above has the same role as the hydrophobic silane compound of the formula (5). The hydrophobic silane compound is exemplified by methyltrimethoxysilane, propyltriacetoxysilane, etc.

The immobilization layer is prepared by coating the primer layer with a coating composition, the coating composition being prepared by dissolving the compound of formula (4) and the optional compound selected from the group consisting of formula (2), formula (5), and mixtures thereof in a dilution solvent.

When the silane compound of formula (4) is mixed with the hydrophobic silane compound selected from the group consisting of formula (2), formula (5), and mixtures thereof, the weight ratio is 0.01:99.99 to 100:0, and preferably 40:60 to 95:5.

The dilution solvent is an organic solvent, water, or a mixture of the organic solvent and water. The organic solvent is preferably an alcohol solvent such as methanol, ethanol, propanol, or butanol; a cellosolve solvent such as methyl cellosolve; any organic solvent compatible with water such as acetones; or any mixture thereof. Since the dilution solvent is an organic solvent compatible with water, the silane oligomer is readily co-polymerized to obtain the coating composition, and is environmentally friendly.

The coating composition for forming the immobilization functional group pattern comprises 0.1 to 90 wt %, preferably 0.1 to 50 wt %, of the immobilization functionalized silane compound. When the amount of the silane compound is less than 0.1 wt %, the immobilization functional group is not sufficiently formed, whereas when it is more than 90 wt %, the coating composition cannot be applied to the substrate.

According to one preferred embodiment of the present invention, the immobilization layer is formed by a coating composition comprising a silane oligomer hydrate and the dilution solvent, wherein the silane oligomer hydrate is obtained by copolymerizing the silane compound having the immobilization functional group in water or a mixed solvent containing water and an organic solvent. The dilution solvent is selected from the group consisting of water, organic solvent, and a mixed solvent of water and an organic solvent.

When an amino silane compound, one of the compounds of formula (4) having an amino group as the immobilization functional group, is polymerized in water, the compound represented by the following formula (6) is obtained:

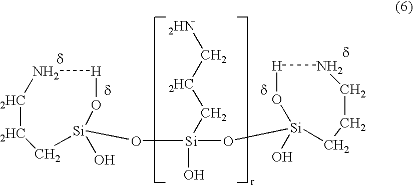

wherein r is the degree of the polymerization.

An amino silane compound of formula (4) wherein the-immobilization functional group is an amino group is polymerized together with the hydrophobic silane compound of formula (5) to provide the amino silane oligomer hydrate represented by the following formula (7):

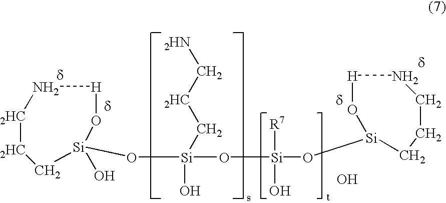

wherein

R⁶ is the same as defined in formula (5), and

s and t are respectively degrees of copolymerization.

In order to increase the reaction rate, any organic or inorganic acid catalyst, such as acetic acid, nitric acid, hydrochloric acid and so on, is added so that the pH of the coating composition is adjusted to a value ranging from 2 to 10. The copolymerization reaction is preferably carried out at a temperature of 0° C. to 100° C. for 1 to 24 hours.

The silane oligomer hydrate maintains a stable reaction equivalent rate so as to not participate in a further reaction since the terminal amino group is bound with the terminal hydroxyl group via a hydrogen bond in the coating composition as shown in formulae (6) and (7).

Further, according to other preferred embodiments of the present invention, the silane compound having the immobilization functional group is dissolved in water or a mixed solvent containing water and an organic solvent so that the silane oligomer hydrate is obtained in the coating composition by the copolymerization reaction.

A desirable immobilization functional group pattern can be formed on the primer layer using the coating composition including a compound with an immobilization functional group. A method for forming the immobilization functional group pattern includes piezoelectric printing using an ink jet printer apparatus, screen printing, micropipeting, and spotting, but it is not limited thereto.

As shown in FIG. 1, through the patterning method, droplets 30 are present on the silanol groups of the primer layer 20. Among the droplets, hydrophobic groups exist to maintain the distance between the droplets and the size of the droplets.

Subsequent to forming the immobilization functional group pattern, the patterned substrate is subjected to heat-treatment. Through this heat-treatment, the coated silane oligomer is thermoset and condensed to provide an immobilization layer having a three dimensional cross-linking structure. Further, the silanol groups of the primer layer 20 are subjected to a condensation reaction with those of the silane oligomer to form a siloxane bond. The heat-treatment temperature is preferably from 100 to 350° C. When the temperature is less than 100° C., the condensation is not sufficient, whereas when the temperature is more than 350° C., the immobilization functional group rapidly degenerates.

The substrate of the present invention having the immobilization functional group pattern comprises a substrate 10; a primer layer 20 formed on the substrate 10 for controlling the surface tension of the upper layer of the immobilization layer, wherein the primer layer has reactive groups to bind an immobilization functional group and hydrophobic functional groups and is thus capable of forming a functional group pattern; and an immobilization layer 30 formed on the primer layer 20 for immobilizing the physiological material. The hydrophobic functional group is preferably a C₁₋₂₀ alkyl, a C₁₋₂₀ haloalkyl, or a C₆₋₁₂ aromatic group, and is more preferably methyl, octyl, heptadecafluoro-1,1,2,2-tetrahydrodecyl, (3-heptafluoroisopropoxy)propyl, or phenyl.

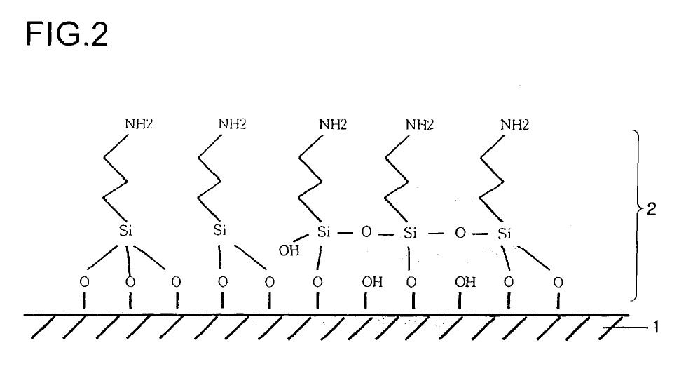

As shown in FIG. 2, the conventional immobilization layer 2 formed on the substrate 1 is a self-assembly monolayer. The self-assembly monolayer is manufactured for an extended duration, and it is difficult to obtain a functional group with a uniform density.

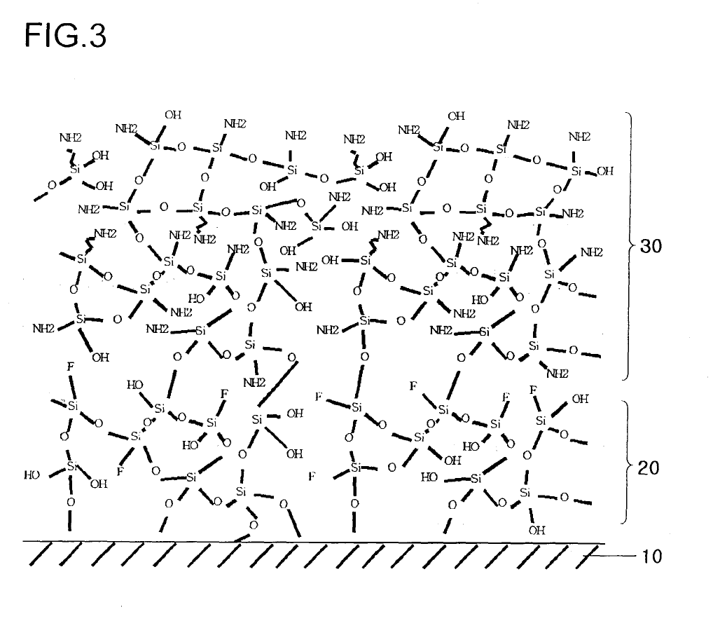

As shown in FIG. 3, the present invention can provide the immobilization layer 30 with a three-dimensional cross-linking structure, so as to provide the functional group uniformly. Further, the immobilization layer with a high-density functional group is fabricated in a relatively short time.

The three dimensional cross-linking structure prevents elimination of the immobilization functional groups and detachment of the physiological material while being washed with solvents used during the immobilization or washing step. Therefore, the thermal stability and reagent stability are improved due to the structural characteristics.

The density of the immobilization groups is determined by analyzing light emitted from fluorescent dye in the immobilization layer upon continuous irradiation of a laser beam, the dye being fluorescein isothiocyanate (FITC), tetraethylrhodamine isothiocyanate (SCN-TMR), or tetramethylrhodamine succinimide (SIE-TMR) which are activated with isothiocyanate or succinimide ether.

The results of the density analysis indicate that the substrate for immobilizing a physiological material according to the present invention has a very stable immobilization functional group at a uniform and high density. For example, the patterned substrate can define arrays of functionalized binding sites of 1 to 10³ per cm² in a diameter of 50 to 5000 micrometers.

The present invention also provides a biochip fabricated by attaching the physiological material to the immobilization functional group on the substrate or by attaching the physiological material activated to have a functional group onto the substrate, and washing out the unreacted physiological material to form a predetermined pattern. The physiological material is preferably reacted with the immobilization layer for 1 to 24 hours.

The term “physiological material” herein means one derived from an organism or its equivalent, or one prepared in vitro. It includes, for example, enzymes, proteins, antibodies, microbes, animal and plant cells and organs, neurons, DNA and RNA, and preferably DNA, RNA, or a protein, wherein the DNA may include cDNA, genome DNA, and an oligonucleotide; the RNA may include genome RNA, mRNA, and an oligonucleotide; and the protein may include an antibody, an antigen, an enzyme, a peptide, etc.

The method for patterning the physiological material on the immobilization layer may be any method of photolithography, piezoelectric printing, micropipeting, spotting, etc.

Hereinafter, the present invention will be explained in detail with reference to examples. These examples, however, should not in any sense be interpreted as limiting the scope of the present invention.

### EXAMPLE 1

3 g of tetraethyl orthosilicate and 0.25 g of heptadecafluoro-1,1,2,2-tetrahydrodecyl)triethoxysilane were added to 90 g of ethanol followed by addition of 7 g of water, and the pH thereof was adjusted to pH 2 by adding nitric acid, to obtain a coating composition for forming a primer layer for controlling the surface tension of an upper layer of an immobilization layer. A glass slide was coated with the coating composition using a spin-coating method to form a primer layer on the glass slide. 5 g of 3-aminopropyltrimethoxysilane were mixed with 15 g of water and reacted at 60° C. for 8 hours to obtain an aminosilane oligomer hydrate. 10 g of the aminosilane oligomer hydrate were dissolved in 90 g of ethanol to provide a coating composition for forming an immobilization layer having a functional group pattern. The coating composition for forming an immobilization layer is piezoelectric-printed using PLOTTER (Trade name: Nano-Plotter, GeSiM) to form a patterned immobilization layer, and then thermoset at 150° C. for 60 minutes, to form a patterned substrate for immobilizing a physiological material.

### EXAMPLE 2

3 g of tetraethyl orthosilicate and 0.25 g of heptadecafluoro-1,1,2,2-tetrahydrodecyl)triethoxysilane were added to 90 g of ethanol followed by addition of 7 g of water, and the pH thereof was adjusted to pH 2 by adding nitric acid, to obtain a coating composition for forming a primer layer for controlling the surface tension of an upper layer of an immobilization layer. A glass slide was dipped into and coated with the coating composition to form a primer layer thereon. 3.55 g of 3-aminopropyltrimethoxysilane and 1.5 g of methyl silane oligomer (Trade name: XS331-B1410, GE Toshiba silicon Co.) were mixed with a mixed dilution solvent including 6 g of water and 6 g of ethanol, the pH thereof was adjusted to pH 9 by adding acetic acid, and the mixture was then reacted at 60° C. for 8 hours to obtain an aminosilane-methylsilane oligomer hydrate. 10 g of the aminosilane-methylsilane oligomer hydrate were dissolved in 90 g of ethanol to provide a coating composition for forming an immobilization functional group pattern. The coating composition for forming an immobilization functional group pattern was piezoelectric-printed using PLOTTER (Nano-Plotter, GeSiM) to form a patterned immobilization layer, and then thermoset at 150° C. for 60 minutes, to form a patterned substrate for immobilizing a physiological material.

### COMPARATIVE EXAMPLE 1

2.5 g of aminopropyltrimethoxysilane were mixed with a mixed dilution solvent including 7.5 g of water and 90 g of ethanol and reacted at 60° C. for 8 hours to obtain an aminosilane oligomer hydrate. 10 g of the aminosilane oligomer hydrate were dissolved in 90 g of ethanol to provide an aminosilane oligomer hydrate-bearing coating composition for forming an immobilization layer. A glass slide was dipped into and coated with the coating composition, and it was then thermoset at 100° C. for 60 minutes to form a substrate for immobilizing a physiological material.

### COMPARATIVE EXAMPLE 2

The patterned substrate for immobilizing a physiological material of this Comparative Example was prepared according to the same process as in U.S. Pat. No. 5,985,551. First, a glass slide was immersed in a mixed solution including 50 g of 3-aminopropyltrimethoxysilane and 15 g of toluene for 20 minutes, and then agitated in toluene for 30 minutes to remove excessive aminopropyltrimethoxysilane, followed by washing twice and drying at 100° C. for 60 minutes to prepare a hydrophilic substrate with an amino group. Subsequently, a blocking surface was formed by reacting the amino group with 4-nitrobenzyl chloroformate as a temporary photolabile blocking material and then exposing the photoblocked substrate surface to light through a mask to create unblocked areas on the substrate surface with an unblocked amino group. The exposed surface of the substrate was reacted with perfluoroacylchloride to form a stable hydrophobic alkyl siloxane matrix. Then, this remaining photoblocked substrate surface was exposed to create patterned regions of the unblocked amino group to produce a patterned substrate having the derivatized hydrophilic binding site regions.

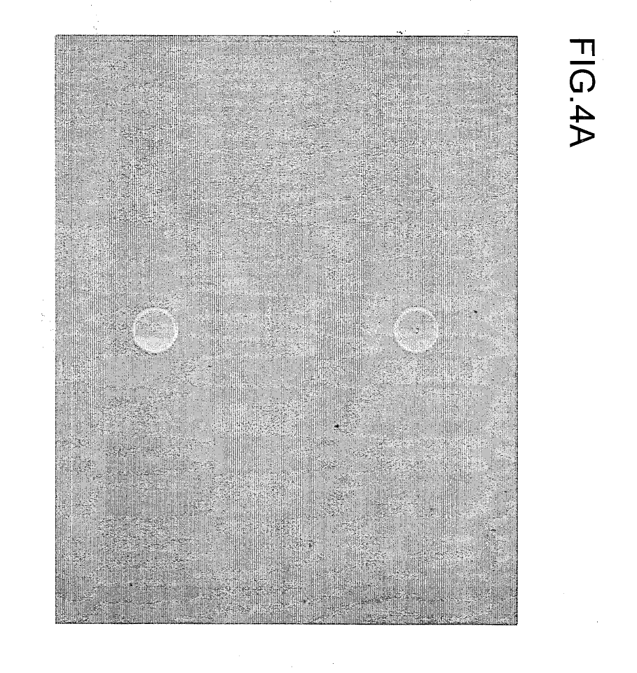

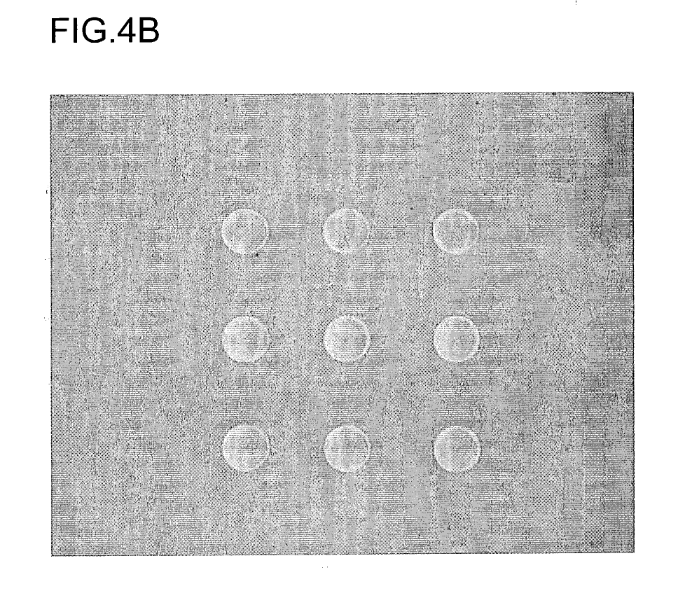

The substrates for immobilizing a physiological material fabricated by the methods according to Examples 1 and 2 of the present invention and Comparative Example 1 were immersed in an aqueous dispersion solution including 5 wt % of Au/Ag colloidal particles (available from Mitsubishi Material. Co.) for 1 minute. FIGS. 4A and 4B are photographs of the substrates of Examples 1 and 2 after immersion. As shown in FIGS. 4A and 4B, in the substrate for immobilizing a physiological material according to Examples 1 and 2, uniform-sized patterns were formed in certain regions, and in other regions, patterns were not formed indicating that immobilization functional groups were not formed in such regions. On the other hand, no pattern was formed on the substrate for immobilizing a physiological material according to Comparative Example 1.

For the substrates for immobilizing a physiological material according to Example 1 and Comparative Example 2, the density of the immobilization functional group was evaluated. The immobilization layers were labeled with a dimethylformamide solution, which was prepared by dissolving FITC in dimethylformamide. A laser beam was continuously irradiated onto the immobilization layer, and the light emitted from the FITC on the layer was detected by a ScanArray 5000 (manufactured by Packard-Biochip Technology Co.). The results of the measurement were as follows: the fluorescence strength of Example 1 was 20,800, whereas that of Comparative Example 2 was 8,000. The fluorescence strength of Example 1 was therefore remarkably superior to that of Comparative Example 2. This indicates that the substrate for immobilizing a physiological material of the present invention has a dense immobilization functional group. These results also indicate that reactivity of the immobilization functional groups was reduced through reaction between the immobilization functional group and the photolabile blocking material, and through the removal of photolabile blocking material.

The present invention can preserve the patterned substrate having a uniform immobilization functional group pattern by providing a primer layer including a reactive group capable of reacting a silanol group of an immobilization layer, and a hydrophobic functional group capable of controlling the surface tension of the immobilization layer.

While the present invention has been described in detail with reference to the preferred embodiments, those skilled in the art will appreciate that various modifications and substitutions can be made thereto without departing from the spirit and scope of the present invention as set forth in the appended claims.

1. A method of preparing a functional-group-patterned substrate for immobilizing a physiological material, comprising:
a) preparing a coating composition including an alkoxide compound and a hydrophobic functionalized silane compound;
b) coating the composition on a substrate to form a primer layer for controlling surface tension of an immobilization layer;
c) forming an immobilization functional group pattern by coating a composition including a compound having a functional group capable of immobilizing the physiological material on the primer-layer-coated substrate to prepare a patterned substrate; and
d) subjecting the patterned substrate to heat-treatment.

2. The method according to claim 1, wherein the alkoxide compound is represented by the following formula (1):
M(OR¹)ₖ (1)
wherein
M is an element selected from the group consisting of 4B, 3A, 4A, and 5A group elements of the Periodic Table;
R¹ is hydrogen or a C₁₋₂₀ alky or C₆₋₁₂ aromatic group; and
k is a value ranging from 3 to 4 and is determined depending upon the valence of M.

3. The method according to claim 1, wherein the hydrophobic functionalized silane compound is represented by the following formula (2):
X—Si(R²)₃ (2)
wherein
X is a hydrophobic functional group; and
R² is hydrogen, C₁₋₂₀ alkyl, or halogen.

4. The method according to claim 3, wherein the hydrophobic functional group is selected from the group consisting of C₁₋₂₀ alkyl, C₁₋₂₀ haloalkyl, and C₆₋₁₂ aromatic groups.

5. The method according to claim 1, wherein the alkoxide compound is a silicon tetraalkoxide.

6. The method according to claim 5, wherein the silicon tetraalkoxide is selected from the group consisting of tetraethyl orthosilicate, aluminum tributoxide, zirconium tetrabutoxide, and mixtures thereof.

7. The method according to claim 1, wherein the hydrophobic functionalized silane compound is selected from the group consisting of (heptadecafluoro-1,1,2,2-tetrahydrodecyl)trialkoxysilane, (heptadecafluoro-1,1,2,2-tetrahydrodecyl)trichlorosilane, (3-heptafluoroisopropoxy)propyl trichlorosilane, and mixtures thereof.

8. The method according to claim 1, wherein the alkoxide compound and the hydrophobic functionalized silane compound are used in a weight ratio of 99.999:0.0001 to 50:50.

9. The method according to claim 1, wherein the coating composition for forming a primer layer further comprises a compound of the following formula (3):
[M′(OR³)ₘ]ₚ(R⁴)_{q} (3)
wherein
M′ is an element selected from the group consisting of 4B, 3A, 4A, and 5A group elements of the Periodic Table;
R³ is hydrogen, halogen, a C₁₋₂₀ alkyl group or a C₆₋₁₂ aromatic group;
R⁴ is a methylene or a phenyl, optionally substituted with a C₁₋₆ substituent;
m is a value ranging from 2 to 3 and is determined depending upon the valence of M′;
p is a numerical value ranging from 2 to 4; and
q is a numerical value ranging from 1 to 20.

10. The method according to claim 9, wherein the compound of the formula (3) is included in an amount of 0.001 to 50 wt % based on the amount of the coating composition.

11. The method according to claim 1, wherein the substrate is selected from the group consisting of glass, silicone wafers, polycarbonate, polystyrene, and polyurethane.

12. The method-according to claim 1, wherein the coating composition to form a primer layer comprises compounds capable of controlling the surface tension of the immobilization layer and a dilution solvent, and the compounds include an alkoxide compound and a hydrophobic functionalized silane compound.

13. The method according to claim 12, wherein the coating composition to form a primer layer comprises 0.1 to 90 wt % of compounds capable of controlling the surface tension of the immobilization layer.

14. The method according to claim 12, wherein the primer layer is formed using a wet coating method selected from the group consisting of dipping, spraying, spin-coating, and printing.

15. The method according to claim 1, wherein the compound having a functional group capable of immobilizing the physiological material is an immobilization functionalized silane compound represented by the following formula (4):
Y-R⁵—Si(R⁶)₃ (4)
wherein
Y varies depending upon the terminal group of the physiological material and is at least one functional group selected from the group consisting of amino, aldehyde, mercapto, and carboxyl groups;
R⁵ is selected from the group consisting of C₁₋₂₀ alkyl groups, C₆₋₂₀ aromatic groups, ester groups, and imine groups; and
R⁶ is selected from the group consisting of hydroxyl groups, C₁₋₂₀ alkoxy groups, acetoxy groups, halogen groups, and combinations thereof.

16. The method according to claim 15, wherein the compound of formula (4) is selected from the group consisting of 3-aminopropyltrimethoxysilane, 3-aminopropyltriethoxysilane, 2-aminoundecyl-trimethoxysilane, aminophenyltrimethoxy-silane, N-(2-aminoethylaminopropyl) trimethoxysilane, 3-mercaptopropyltrimethoxy-silane, 3-mercaptopropyltriethoxysilane, 4-trimethoxysilylbutanal, 4-trimethoxy-silylbutanal, carboxymethyltrimethoxysilane, carboxymethyltriethoxysilane, and mixtures thereof.

17. The method according to claim 1, wherein the composition including a compound having a functional group capable of immobilizing the physiological material further comprises:
a hydrophobic functionalized silane compound that is represented by the following formula (2); a hydrophobic silane compound represented by the following formula (5); and mixtures thereof:
X—Si(R²)₃ (2)
wherein:
X is a hydrophobic functional group; and
R² is hydrogen, C₁₋₂₀ alkyl, or halogen, 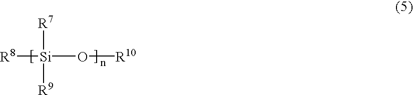
wherein
R⁷ is selected from the group consisting of C₁₋₁₄ alkyl groups, C₆₋₁₂ aromatic groups optionally substituted with methyl, ethyl or propyl, and CX₃, wherein X is a halogen;
R⁸ and R⁹ are each independently selected from the group consisting of C₁₋₁₄ alkoxy groups, acetoxy groups, hydroxyl groups, and halogen groups;
R¹⁰ is selected from the group consisting of hydrogen, C₁₋₁₄ alkyl groups, and C₆₋₁₂ aromatic groups; and
n is an integer ranging from 1 to 15.

18. The method according to claim 1, wherein the immobilization functional group pattern is formed using a method selected from the group consisting of piezoelectric printing, screen printing, micropipeting, and spotting.

19. The method according to claim 15, wherein the coating composition for forming the immobilization functional group pattern comprises 0.1 to 90 wt % of the immobilization functionalized silane compound.

20. The method according to claim 1, wherein the heat-treatment of the patterned substrate is performed at a temperature ranging from about 100° C. to about 350° C.

21. A substrate having an immobilization functional group pattern for immobilizing a physiological material, wherein the substrate is fabricated by the processes comprising:
a) preparing a coating composition including an alkoxide compound and a hydrophobic functionalized silane compound;
b) coating the composition on a substrate to form a primer layer for controlling surface tension of an immobilization layer;
c) forming an immobilization functional group pattern by coating a composition including a compound having a functional group capable of immobilizing the physiological material on the primer-layer-coated substrate to prepare a patterned substrate; and
d) subjecting the patterned substrate to heat-treatment.

22. A substrate with an immobilization functional group pattern comprising
a) a substrate;
b) a primer layer formed on the substrate for controlling surface tension of an upper layer of an immobilization layer, wherein the primer layer has reactive groups to bind with an immobilization functional group and hydrophobic functional groups capable of controlling functional group patterning; and
c) a patterned immobilization layer formed on the primer layer for immobilizing the physiological material.

23. The substrate according to claim 22, wherein the hydrophobic functional group is selected from the group consisting of C₁₋₂₀ alkyl groups, C₁₋₂₀ haloalkyl groups, and C₆₋₁₂ aromatic groups.

24. The substrate according to claim 22, wherein the patterned substrate defines arrays of functionalized binding sites of 1 to 10³ per cm² in a diameter of 50 to 5000 micrometers.

25. A biochip comprising an immobilized physiological material, wherein the biochip is fabricated by binding physiological material or activated physiological material having a functional group on a surface of the patterned substrate according to claim 22, followed by washing to remove unbound physiological material to form a physiological material pattern.

26. The biochip according to claim 25, wherein the physiological material is selected from the group consisting of enzymes, proteins, DNA, RNA, microbes, microorganisms, animal and plant cells and organs, and neurons.

27. The biochip according to claim 25, wherein the physiological material pattern is formed using a method selected from the group consisting of piezoelectric printing, screen printing, micropipeting, and spotting.
